(19) United States
(12) Patent Application Publication
(10) Pub. No.: US 2022/0290087 A1
(43) Pub. Date: Sep. 15, 2022

(54) CELL CO-CULTURE SYSTEM AND METHOD

(71) Applicants: FONDAZIONE ISTITUTO ITALIANO DI TECNOLOGIA, (US); SCUOLA UNIVERSITARIA SUPERIORE SANT'ANNA, (US)

(72) Inventors: Attilio MARINO, Cascina (IT); Daniele DE PASQUALE, Marsala (IT); Edoardo SINIBALDI, Pisa (IT); Gianni CIOFANI, Monterosso al Mare (IT)

(73) Assignees: FONDAZIONE ISTITUTO ITALIANO DI TECNOLOGIA, Genova (IT); SCUOLA UNIVERSITARIA SUPERIORE SANT'ANNA, Pisa (IT)

(21) Appl. No.: 17/768,195

(22) PCT Filed: Oct. 6, 2020
(86) PCT No.: PCT/IB2020/059365

§ 371 Date: Apr. 11, 2022

(30) Foreign Application Priority Data
Oct. 11, 2019 (IT) 102019000018614

(51) Int. Cl. C12M 1/12; C12M 1/42; C12N 5/00

(52) U.S. Cl. CPC C12M 25/14; C12M 35/06; C12N 5/0068; C12N 2529/00; C12N 2535/00

(57) ABSTRACT

The present invention relates to a method and an apparatus for in vitro three-dimensional cell co-culture, wherein said method comprises a step of seeding a plurality of cells of a first cell type on a first magnetic prismatic porous scaffold and a plurality of cells of a second cell type on a second magnetic prismatic porous scaffold, while keeping the first and second scaffolds physically separate, and a step of moving the first and second scaffolds towards each other under the action of a magnetic field generated by a magnetic field generator until contact occurs on at least one surface.

The present invention relates to a system and a method for in vitro three-dimensional cell co-culture.

As is known, cell-to-cell and cell-to-matrix interactions play a fundamental role in biological tissues for cell survival, growth, proliferation, migration and differentiation. In particular, cell-to-cell interactions are regulated via secretion of signalling molecules and direct contact among the cells.

Unlike cellular monocultures, multicellular co-culture systems allow interaction among cells of different populations within the system. For this reason, such systems can be used for in vitro reproduction of complex biological structures, wherein two or more different cellular populations interact with each other. For example, such systems may be used for modelling biological structures, such as neuromuscular junctions and pancreatic islets, or pathological states of the organism, such as infiltration of tumor cells in healthy tissues.

The co-culture systems that are currently available can be divided into “direct” and “indirect” co-culture systems. Indirect co-culture systems are those which permit the exchange of biochemical signals, but not direct contact, among the cells. For example, patent application WO2007021919 shows a multi-chambered co-culture system wherein cells of different populations are divided in separate chambers and communicate only by exchanging signalling molecules through a semipermeable membrane. It is clear that systems of this kind can reproduce the natural interaction among the cells only partially, since they completely exclude any phenomena related to the physical interfacing among the cells.

Conversely, direct co-culture systems can reproduce the interaction among different cells in a more complete manner because they also allow physical contact among cells belonging to different populations. However, the systems currently known in the art are based on co-culture of cells of distinct populations on the same support substrate, e.g. on three-dimensional porous structures commonly known in the tissue engineering field as “scaffolds”, or on the overlapping of single layers of cells belonging to different populations in a “sandwich” fashion (as shown, for example, by Suhaeri et al. in the scientific publication “Novel Platform of Cardiomyocyte Culture and Coculture via Fibroblast-Derived Matrix-Coupled Aligned Electrospun Nanofiber”, ACS applied materials & interfaces 9.1 (2016): 224-235).

The systems based on the seeding of cells of different populations on the same substrate do not permit selecting when to join the different cell populations within the co-culture system. This prevents, for example, separate cultivation of three-dimensional structures of each cell type by following a strictly type-related protocol; for example, it is not possible to select specific support substrates and cell culture media for each cell type. Moreover, such systems do not permit obtaining three-dimensional structures with a predefined spatial distribution of the cells.

On the other hand, sandwich-type co-culture systems do not permit developing complex three-dimensional structures capable of accurately reproducing the biological structures of organs and apparatuses.

In light of the above examination, it is therefore a technical problem at the basis of the invention to provide a system and a method for cell co-culture having such features that allow overcoming the limitations of the above-described state of the art.

The present invention aims at solving this and other problems by providing an in vitro cell co-culture method exploiting the magnetic interaction between the scaffolds.

In addition, the present invention aims at providing an in-vitro cell co-culture system which makes it possible to implement the method.

The idea that solves the aforesaid problem is to effect the interfacing between three-dimensional structures of cells of different types, grown on magnetic scaffolds, via magnetic interaction between said scaffolds in the presence of a magnetic field.

The solution proposed herein permits a stable interfacing between three-dimensional structures of cells of different types, thus making it possible to create, in a simple manner, complex three-dimensional structures in which at least two cell types coexist.

Further advantageous features of the present invention will be set out in the appended claims.

Furthermore, the fundamental features as well as further advantages of the present invention will become more apparent from the following description of a preferred, but non-exclusive, embodiment thereof as shown in the annexed drawings, which are supplied merely by way of non-limiting example, wherein:

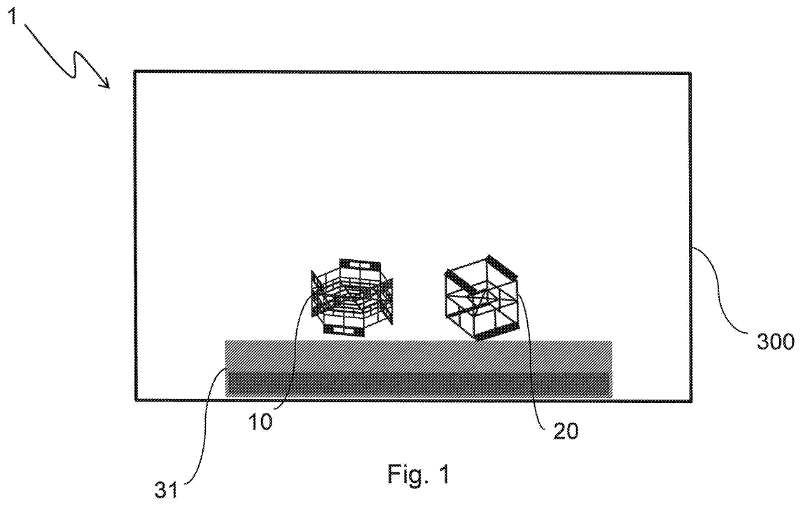

FIG. 1 shows a schematic representation of a system according to the invention;

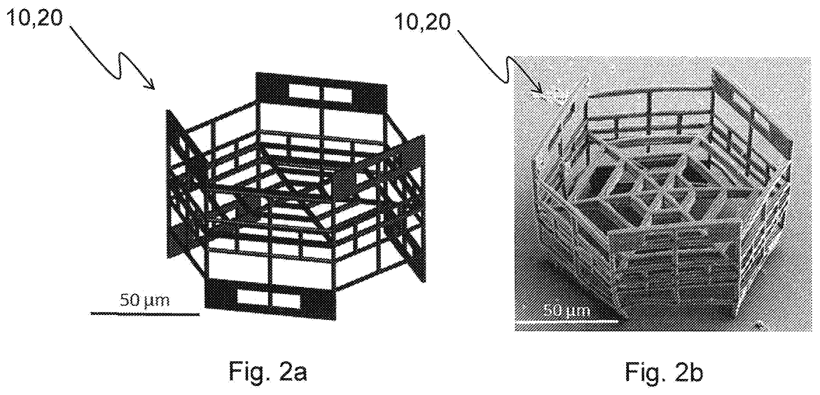

FIG. 2a shows a representation of a scaffold according to a preferred solution;

FIG. 2b shows an image taken with a scanning electron microscope (SEM) of a scaffold according to a preferred solution;

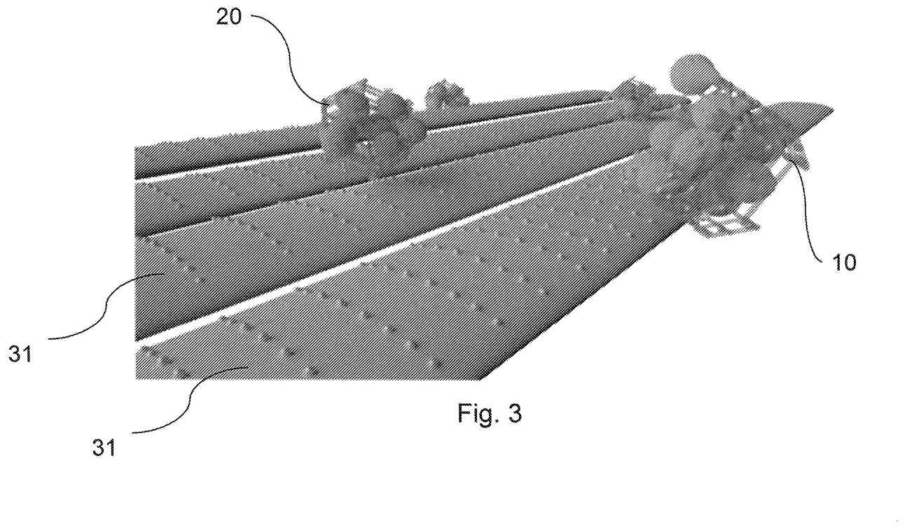

FIG. 3 shows a schematic representation of a part of the system of the invention in accordance with a preferred solution;

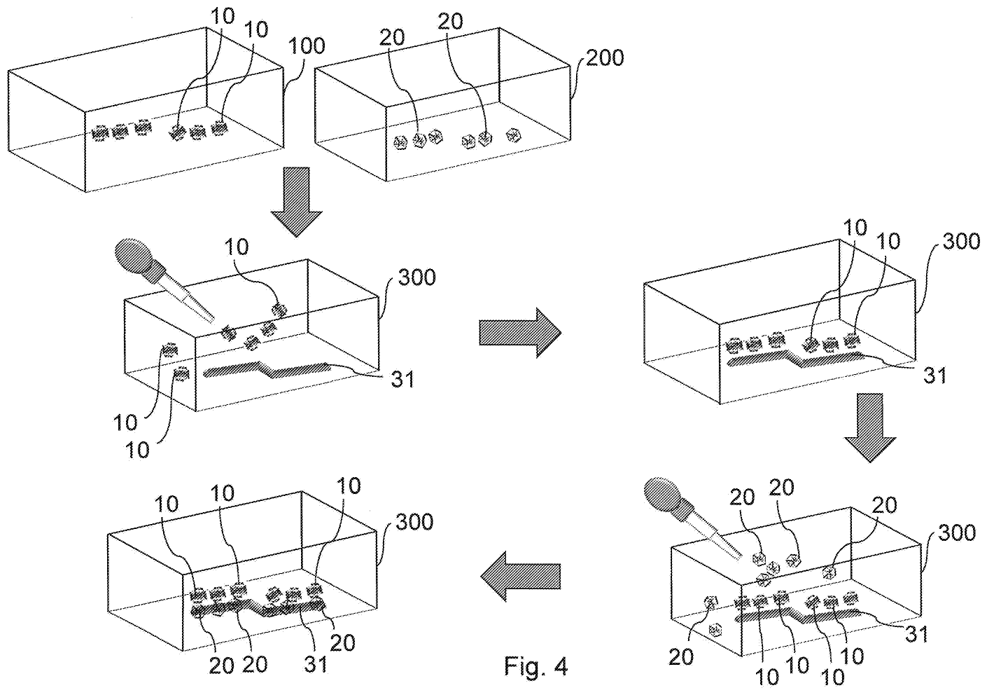

FIG. 4 schematically illustrates the steps of the method according to a preferred solution;

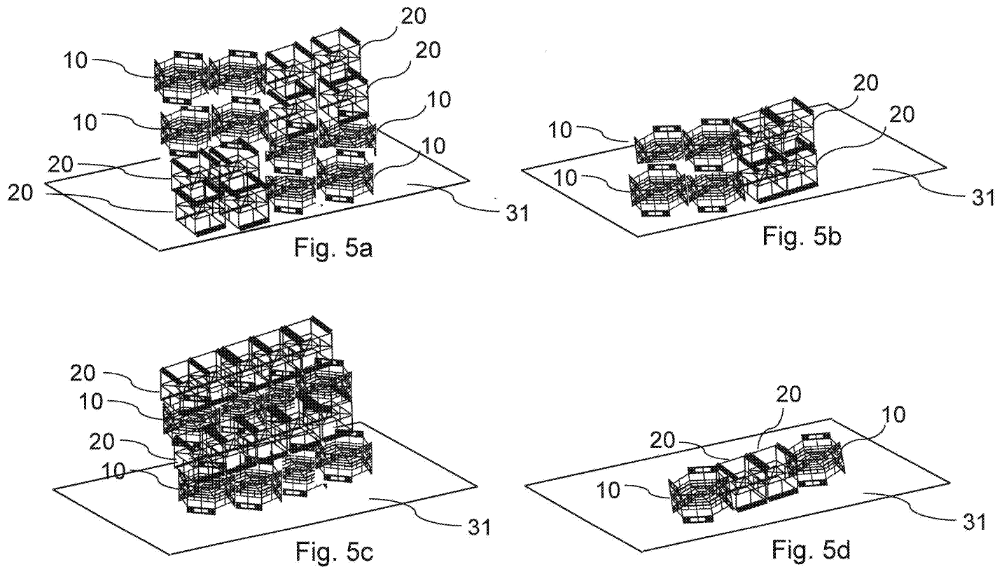

FIGS. 5a, 5b, 5c and 5d schematically illustrate some possible configurations of the scaffolds within the co-culture system according to the invention;

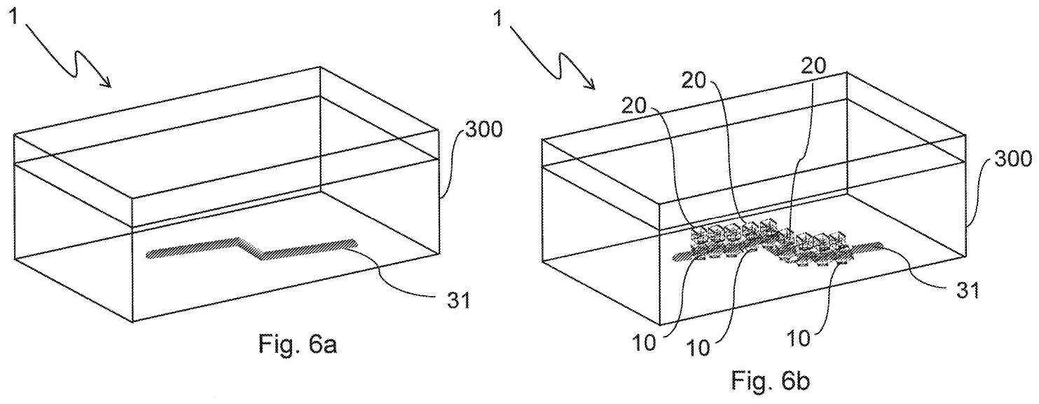

FIGS. 6a and 6b schematically illustrate a preferred embodiment of the system according to the invention;

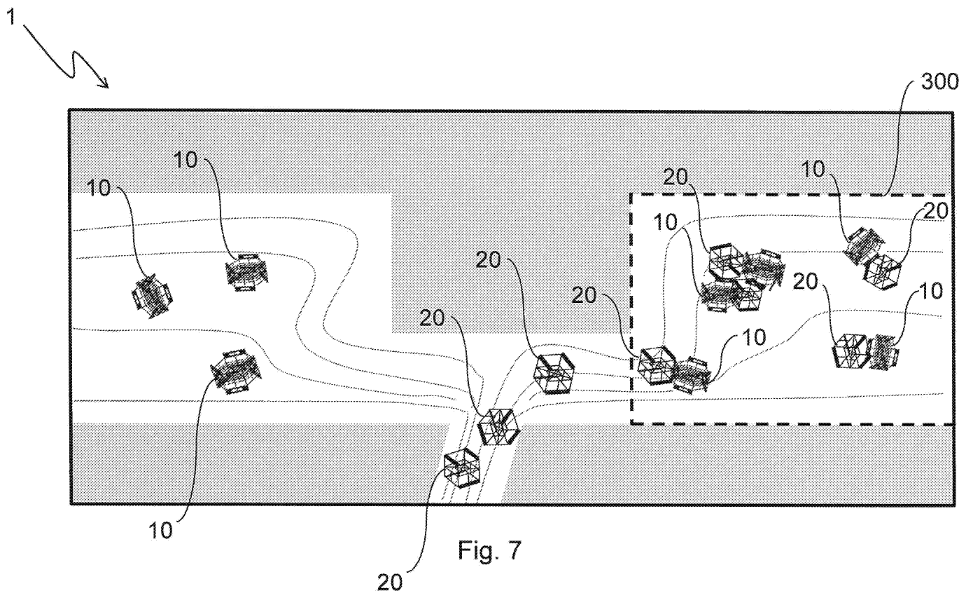

FIG. 7 schematically illustrates a preferred embodiment of a part of a system in accordance with the invention and the method.

Before proceeding any further with a detailed description, it must be pointed out that any reference to “an embodiment” or “an implementation” in this description will indicate that a particular configuration, structure or feature is comprised in at least one embodiment of the invention. Therefore, the expression “in one embodiment” and the like, which can be found in different parts of this description, will not necessarily refer to the same embodiment. Moreover, any particular configurations, structures or features may be combined as deemed appropriate in one or more embodiments. The references below are therefore used only for simplicity's sake and shall not limit the protection scope or extent of the various embodiments.

In light of this introductory statement, and with reference to FIG. 1, a system 1 for in vitro cell co-culture according to the invention comprises at least one first scaffold 10 comprising magnetizable material suitable for supporting the growth of cells of a first cell type, at least one second scaffold 20 comprising magnetizable material suitable for supporting the growth of cells of a second cell type, a magnetic field generator 31 adapted to generate a magnetic field that produces a magnetic attraction force between the first scaffold 10 and the second scaffold 20, a cell co-culture chamber 300, in which the scaffolds 10,20 are subjected to the magnetic field generated by the magnetic field generator 31 and approach each other until they generate at least one interface surface.

The scaffolds 10,20 comprise magnetizable material, and are therefore susceptible of magnetization, i.e. they can become magnetized when subjected to the action of a magnetic field. Preferably, the scaffolds 10,20 comprise at least one material having ferromagnetic properties and/or at least one material having superparamagnetic properties.

The scaffolds 10,20 have selected structural characteristics for supporting the growth of each cell type being co-cultured, as described in the literature; for example, they may have characteristics like those described by Nava et al. in the scientific publication “3D Stem Cell Niche Engineering via Two-Photon Laser Polymerization”, Methods in Molecular Biology 1612 (2017): 253.

Said scaffolds 10,20 are three-dimensional porous structures created by using scaffold production techniques known in the tissue engineering field (e.g. multi-photon polymerization, 3D printing, electrospinning, injection moulding, etc.).

Preferably, the scaffolds 10,20 are made of biocompatible polymeric material (e.g. collagen) or hybrid polymeric/ceramic material (e.g. Ormocomp®, produced by Nanoscribe GmbH), and comprise magnetic material (e.g. in the form of nanoparticles, thin coating, or the like) for the purpose of making the scaffolds 10,20 susceptible of magnetization.

Preferably, the scaffolds 10,20 have a substantially prismatic shape; for example, they are prisms with a base having a concave or convex simple polygonal shape (e.g. prisms with a square, rectangular or hexagonal base, or with a base having a more complex shape).

In a preferred embodiment, the first scaffolds 10 and the second scaffolds 20 have different shapes, so as to permit discerning the cell cultures within the co-culture system 1 without having to use dyes or other tracers on the cells.

Preferably, the first scaffold 10 and the second scaffold 20 have complementary shapes, for the purpose of promoting the mutual anchoring of the scaffolds.

The magnetic field generator 31 in the co-culture chamber 300 is, for example, a permanent magnet or an electromagnet. Preferably, the magnetic field generator 31 is an element of magnetized ferromagnetic material (in the form of a plate, a leaf, a cylinder, or the like) constrained to the co-culture chamber 300. More preferably, the magnetic field generator 31 is directly integrated into at least one of the scaffolds 10,20; for example, the magnetic field generator 31 is integrated into the structure of at least one of the scaffolds 10,20 as nanoparticles of magnetic material included in the polymeric matrix of the scaffolds 10,20. The nanoparticles may be, for example, beads of ferromagnetic material having a diameter in the range of 50 to 100 nm, or beads of superparamagnetic material having a diameter in the range of 3 to 20 nm.

The co-culture chamber 300 may be a vessel or a cell culture plate, or may be defined as a delimited space inside a more complex structure (e.g. a microfluidic chip) connected to other environments through suitable connection ways.

Furthermore, the co-culture system 1 may comprise additional culture chambers, whether physically separate from or in communication with the co-culture chamber 300.

### EXAMPLE 1

In a first variant of the invention, the co-culture system 1 comprises at least one first scaffold 10 and at least one second scaffold 20 with superparamagnetic properties and an electromagnetic field generator 31 with ferromagnetic properties.

As shown in FIG. 2, the scaffolds 10,20 are prismatic structures with a hexagonal base, which comprise a horizontal central support structure and vertical lateral support structures.

The horizontal support structure comprises four equidistant concentric hexagonal rings (wherein the sides of the major hexagonal ring have a length of 60 μm) mutually connected by diagonal elements joining the vertices of the hexagons at the centre thereof.

The lateral support structures are square grids (with sides of 60 μm) made up of vertical and horizontal support elements so arranged as to define a plurality of rectangular apertures, and a rectangle of material comprising two rectangular apertures. As can be seen in FIG. 2, the lateral support structures comprise eight minor rectangular apertures in the central part, with sides of 30 μm and 7.5 μm, and four major rectangular apertures (two above and two under the eight minor apertures), with a major side of 30 μm and a minor side of 15 μm; the rectangle of material comprising two rectangular apertures is alternately positioned at the top or at the bottom of the lateral support structure; its sides are 30 μm and 15 μm long and the sides of the apertures are 30 μm and 7.5 μm long.

The scaffolds 10,20 are made of composite material obtained by inserting superparamagnetic nanoparticles into negative photoresist sensitive to UV rays; in particular, 10 mg of superparamagnetic nanoparticles of iron oxide (also known as “SPIONs”) are evenly mixed into 1 ml of IP-L 780 negative photoresist (produced by Nanoscribe GmbH) via a sonication process at 8 W for 2 minutes. The superparamagnetic nanoparticles are beads having a diameter of 3 nm; in general, the allowable diameter of the nanoparticles may range from 3 to 20 nm.

The scaffolds 10,20 are obtained by two-photon polymerization, also known as “direct laser writing”, of the superparamagnetic resist deposited on a glass substrate coated with a sacrificial layer of polyvinyl alcohol (PVA). The PVA sacrificial layer is obtained by depositing on the glass substrate 0.5 ml of PVA at a concentration of 2 mg/ml brought to a temperature of 80° C. for 5 minutes.

Two-photon polymerization of the scaffolds 10,20 is accomplished by using a direct-writing laser lithography system (Photonic Professional System available from Nanoscribe GmbH) with 63× immersion lens, numerical aperture (NA) of 1.4, 780 nm laser beam, writing speed of 10 mm/s and laser power of 70.2 mW. The scaffolds were developed in SU-8 developer (MicroChem Corp.) for 30 minutes and washed with isopropyl alcohol and deionized water, so as to ensure removal of non-hardened photoresist.

The magnetic field generator 31 is an elongate plate made by two-photon polymerization of photoresist including magnetized ferromagnetic particles (as shown in FIG. 3).

### EXAMPLE 2

In a second example, the co-culture system 1 comprises at least one first scaffold 10 with superparamagnetic properties and at least one second scaffold 20 with ferromagnetic properties.

The at least one first scaffold 10 is made of composite material obtained by inserting superparamagnetic nanoparticles into negative photoresist sensitive to UV rays, as described in Example 1.

The at least one second scaffold 20 is made of composite material obtained by inserting magnetized ferromagnetic nanoparticles into negative photoresist sensitive to UV rays, in accordance with the technique described in Example 1, but the iron oxide superparamagnetic nanoparticles are replaced with iron oxide ferromagnetic nanoparticles having a diameter of 50 nm. The allowable diameter of the nanoparticles may however range from 50 to 100 nm. In this example, the second scaffold 20 acts as a magnetic field generator and can cause the magnetization of the superparamagnetic nanoparticles of the first scaffold 10.

### EXAMPLE 3

In a further example, the co-culture system 1 comprises at least one first scaffold 10 and at least one second scaffold with ferromagnetic properties. Both scaffolds 10,20 are made of composite material obtained by inserting ferromagnetic nanoparticles into negative photoresist sensitive to UV rays, in accordance with the same technique described in Example 1, but the iron oxide superparamagnetic nanoparticles are replaced with iron oxide ferromagnetic nanoparticles having a diameter of 50 nm. The allowable diameter of the nanoparticles may however range from 50 to 100 nm.

In this variant, due to the magnetized ferromagnetic nanoparticles, the scaffolds 10,20 act as magnetic field generators 31.

Further variations of the system according to the invention are of course possible. The present invention is not therefore limited to the illustrative examples described herein, but may be subject to many modifications, improvements or replacements of equivalent parts and elements without departing from the basic inventive idea, as specified in the claims.

The following will describe a method for in vitro three-dimensional cell co-culture according to the invention.

The method according to the invention comprises the following steps:
cultivating a plurality of cells of a first cell type on at least one first scaffold 10 comprising magnetizable material and a plurality of cells of a second cell type on at least one second scaffold 20 comprising magnetizable material, while keeping said first and second scaffolds physically separate;
allowing the scaffolds 10,20 to approach each other under the action of a magnetic field, until contact occurs on at least one interface surface.

In other words, the method is based on separate cultivation of at least two different cell cultures on magnetic scaffolds 10,20, followed by union of the two cell cultures by means of a magnetic field, generated by a magnetic field generator 31, which causes the scaffolds 10,20 to move towards each other until complete interfacing is achieved on at least one surface.

This makes it possible to separately grow single cell cultures and then assemble three-dimensional co-culture structures.

The above-described method can be implemented by using systems 1 like those previously illustrated herein.

In one example of embodiment, schematically represented in FIG. 4, the cells of the first cell type are seeded on at least one scaffold 10, within a first culture chamber 100, and the cells of the second cell type are seeded on at least one scaffold 20, within a second culture chamber 200, which is separate from the first culture chamber 100. Subsequently, the scaffolds are transferred into the co-culture chamber 300 and left to interact under the action of the magnetic field inside the chamber.

By releasing the scaffolds 10,20 into the co-culture chamber 300 according to different release sequences, it is possible to obtain scaffold aggregates 10,20 with different scaffold distribution, number of layers, aspect ratio and shape (some examples are shown in FIG. 5). A magnetic field generator element 31 (e.g. a leaf of magnetized ferromagnetic material) constrained to the bottom of the co-culture chamber 300 may be used to define the basic shape of the scaffold aggregate 10,20 (a schematic example is represented in FIGS. 6a and 6b).

In a further example of embodiment, shown in FIG. 7, the scaffolds 10,20 are inserted into a microfluidic chip. The at least one first scaffold 10 and the at least one second scaffold 20 are initially positioned into separate chambers of the microfluidic chip, connected to the co-culture chamber 300 by means of passage ways; subsequently, by adjusting the flow through such ways, the scaffolds are conveyed into the co-culture chamber 300 and interact under the action of the magnetic field.

1. System (1) for in vitro three-dimensional cell co-culture, characterized in that it comprises
a first prismatic porous scaffold (10) comprising magnetizable material suitable for supporting the growth of a first cell type;
a second prismatic porous scaffold (20) comprising magnetizable material suitable for supporting the growth of a second cell type, said second prismatic porous scaffold (20) having a shape complementary to the shape of the first prismatic porous scaffold (10);
a magnetic field generator (31) adapted to generate a magnetic field that produces a magnetic attraction force between the first scaffold (10) and the second scaffold (20);
a cell co-culture chamber (300), in which said first and second scaffolds (10,20) are subjected to the magnetic field generated by the magnetic field generator (31).

2. System (1) according to claim 1, wherein the magnetizable material comprised in the first scaffold (10) and/or in the second scaffold (20) is a ferromagnetic or superparamagnetic material.

3. System (1) according to claim 1 or 2, wherein the magnetic field generator (31) comprises at least one permanent magnet.

4. System (1) according to claim 3, wherein the at least one permanent magnet is an element of magnetized ferromagnetic material having an elongate shape and being substantially as wide as the scaffolds (10,20).

5. System (1) according to claim 1, 2 or 3, wherein the magnetic field generator (31) is comprised in the structure of at least one of the scaffolds (10,20).

6. System (1) according to claim 5, wherein the magnetic field generator (31) comprises a plurality of magnetized ferromagnetic nanoparticles.

7. System (1) according to claim 1, 2 or 3, wherein the magnetic field generator (31) coincides with at least one of the scaffolds (10,20).

8. System (1) according to any one of the preceding claims, comprising a first culture chamber (100) for at least one first scaffold (10) and a second culture chamber (200) for at least one second scaffold (20), wherein said first and second scaffolds (10,20) are not affected by any mutual magnetic attraction force.

9. System (1) according to claim 8, wherein the first culture chamber (100) and the second culture chamber (200) communicate with the co-culture chamber (300).

10. System (1) according to any one of the preceding claims, wherein the co-culture chamber (300) is comprised within a microfluidic chip.

11. Method for in vitro three-dimensional cell co-culture, comprising the steps of
seeding a plurality of cells of a first cell type on a first prismatic porous scaffold (10) comprising magnetizable material suitable for supporting the growth of said first cell type and a plurality of cells of a second cell type on a second prismatic porous scaffold (20) comprising magnetizable material suitable for supporting the growth of said second cell type, said second prismatic porous scaffold (20) having a shape complementary to the shape of the first prismatic porous scaffold (10), while keeping said first scaffold (10) physically separate from said second scaffold (20);
allowing the first scaffold (10) to move towards the second scaffold (20) under the action of a magnetic field generated by a magnetic field generator (31), until contact between said first and second scaffolds (10,20) occurs on at least one surface.

12. Method according to claim 11, wherein the magnetizable material comprised in the first scaffold (10) and/or in the second scaffold (20) is a ferromagnetic or superparamagnetic material.

13. Method according to claim 11 or 12, wherein the magnetic field generator (31) comprises at least one permanent magnet.

14. Method according to claim 13, wherein the magnetic field generator (31) comprises at least one element of magnetized ferromagnetic material having an elongate shape and being substantially as wide as the scaffolds (10,20).

15. Method according to claim 11, 12 or 13, wherein the magnetic field generator (31) is comprised within at least one of the scaffolds (10,20).

16. Method according to claim 15, wherein the magnetic field generator (31) is comprised within at least one of the scaffolds (10,20) as a plurality of magnetized ferromagnetic nanoparticles.

17. Method according to claim 11, wherein the electromagnetic field generator (31) coincides with at least one of the scaffolds (10,20).

18. Method according to any one of the preceding claims, wherein the step of moving the first scaffold (10) and the second scaffold (20) towards each other occurs inside a cell co-culture chamber (300).

19. Method according to claim 18, wherein the cell co-culture chamber (300) is comprised within a microfluidic chip.
